(19) United States
(12) Patent Application Publication
(10) Pub. No.: US 2019/0345185 A1
(43) Pub. Date: Nov. 14, 2019

(54) CONTINUOUS PROCESS FOR HYDROGENATION OF MALTOSE TO MALTITOL

(71) Applicant: Archer-Daniels-Midland Company, Decatur, IL (US)

(72) Inventors: Paul Bloom, Forsyth, IL (US); Kevin Martin, Mt. Zion, IL (US); Chi Cheng Ma, Champaign, IL (US)

(73) Assignee: Archer-Daniels-Midland Company, Decatur, IL (US)

(21) Appl. No.: 16/472,014

(22) PCT Filed: Dec. 19, 2017
(86) PCT No.: PCT/US17/67208

§ 371 Date: Jun. 20, 2019

Related U.S. Application Data
(60) Provisional application No. 62/436,554, filed on Dec. 20, 2016.

(51) Int. Cl. C07H 15/04; C07H 1/00

(52) U.S. Cl. CPC C07H 15/04; C07H 1/00

(57) ABSTRACT

A process for preparing maltitol is described. The process enables greater control of the reaction to minimized generation of glucose and sorbitol side products that can arise from the over-hydrogenation of maltitol. The process involves reacting a medium containing maltose at a concentration of ≤30% with hydrogen in a continuous manner using a fixed-bed reactor at a reaction temperature and pressure sufficient to produce a final product containing a yield of maltitol of at least 90 mol. %, at a conversion rate in which no more than about 2 mol. % of said maltose concentration remains, and a sorbitol concentration of less than 1.0 mol. %.

### FIELD OF INVENTION

The present disclosure relates to a process for preparing a sugar alcohol. In particular, the present process improves conventional methods of making maltitol from maltose.

### BACKGROUND

Maltitol is a sugar alcohol (a polyol) with about 75%-90% of the sweetness of sucrose and nearly identical properties. Food manufacturers have used maltitol as a substitute for table sugar because is lower in calorie content (by about half as combined glucose and fructose), and does not promote tooth decay, and has a lesser effect on raising blood glucose. It is used in several commercial products under trade names such as LESYS™, MALTISWEET™ and SWEETPEARL™.

In terms of chemistry, maltitol is a disaccharide known as 4-O-α-glucopyranosyl-D-sorbitol, which is prepared by the hydrogenation of maltose obtained from starch. Scheme A is a schematic representation of the hydrogenation reaction.

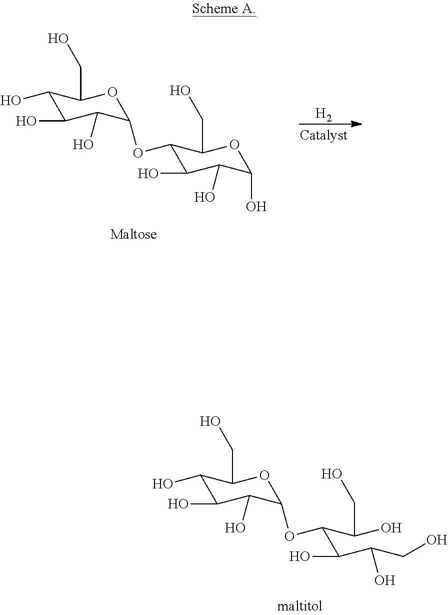 [SMILES: OCC1OC(OC2C(CO)OC(O)C(O)C2O)C(O)C(O)C1O ; "H2" ; OCC(O)C(O)C(OC1OC(CO)C(O)C(O)C1O)C(O)CO]

The hydrogenation reaction opens the maltose ring having a sugar functional group, which converts the sugar moiety to a hydroxyl group and transforms the maltose molecule into maltitol, having a sorbital substituent bound to a glucose substituent.

In conventional processes for making maltitol, one typically employs a high m hose syrups, with over 60% maltose and a minimum of dextrose. However, the use of conventional maltose hydrogenation for producing maltitol is not efficient, due to residual reducing sugar and sorbitol content in the final product. Thus, to improve the batch process, more careful reaction conditions with more process control are needed.

Some examples of preparing conventional high maltose syrups is described in U.S. Pat. Nos. 3,795,584 and 3,804,715, assigned to Hayashibara, which show the saccharification of liquified starch to produce high maltose solutions using beta-amylase and alpha-1,6-glucosidase (pullulanase). Such sugar compositions, however, contain a significant percentage of glucose, which in order to achieve a more pure product should be minimized. U.S. Patent Application Publication No. 2015/0251980 shows that well-mixed slurry reactors (whether operated continuously or, as is more common, in a batch-wise manner) facilitate effective temperature control within the reactor and are characterized by intensive mass transfer between all phases. Well-mixed slurry reactors employing such techniques, however, as admitted in the reference, pose difficulties in terms of the separation of the product from the catalyst due to catalyst attrition, in terms of abrasion of equipment surfaces by moving catalyst particles, in terms of low specific productivity per unit volume, and, for the continuous mode, catalyst separation and regeneration. Alternatively, fixed bed reactors are viewed as more favored, especially for continuous processes. But nonetheless, the fixed-bed reactors have their own limitations, complexities and disadvantages in the context of carrying out multiphase processes involving reactants in gas and liquid phases with a solid catalyst.

In view of the shortcomings of current process protocols and techniques, better and easier methods to making maltitol are warranted. The present invention can provide a pathway by which maltose can be transformed into maltitol with maximized conversion, product yield and minimized side product generation.

### SUMMARY OF THE INVENTION

The present disclosure pertains to a process for preparing maltitol from a maltose-containing syrup. The process involves reacting a medium containing maltose at a concentration of less than or equal to 30% with hydrogen in a continuous manner using a fixed-bed reactor at a reaction temperature and pressure sufficient to produce a final product containing a yield of maltitol of at least 90 mol. %, and a sorbitol concentration of less than 1.0 mol. %. The concentration of hydrogen to maltose substrate is in a molar ratio of ≥20:1. The process is efficient to control the level of sorbitol and dextrose byproducts in the final product, and to consume at least 98 mol. % of the maltose feedstock, such that no more than about 2 mol. % of the concentration of maltose remains at the end of the process. The reactants are processed within the reactor at a liquid hourly space velocity (LHSV) that is ≤3.0 per hour, typically in a range from about 0.1 to 2.8 per hour.

Additional features and advantages of the present process will be disclosed in the following detailed description. It is understood that both the foregoing summary and the following detailed description and examples are merely representative of the invention, and are intended to provide an overview for understanding the invention as claimed.

### BRIEF DESCRIPTION OF FIGURES

FIG. 1 is a schematic of a conventional batch reactor system.

FIG. 2 is a schematic of a continuous reactor system according to an embodiment of the present invention.

### DETAILED DESCRIPTION OF THE INVENTION

The present process enables an operator greater control of the hydrogenation process to convert maltose to maltitol by means of controlling for a combination, in part, of feedstock concentration, ratio of hydrogen to feedstock substrate concentration, hydrogen solubility in a solvent matrix, and space velocity of the reactor. An advantageous feature of the present process is that it allows for a “tunable” system in which an operator can make adjustments to the flow-rate, pressure and temperatures to help reduce overall dwell time of the feedstock in the continuous reactor, and to minimize generating significant amounts of side products through overreacting the product mix. The process can demonstrate ≥98% conversion of maltose to maltitol.

Unlike conventional process reactions, the present process performs in a more uniform manner and does not suffer adverse side-effects associated with over “cooking” the feedstock in hot spots while trying to react the materials in other cooler areas of the reactor. This kind uneven heating and reaction in conventional systems results typically in disassociation of a maltitol molecule, and formation of significant amounts of glucose and sorbitol. The present process employs a lower sugar concentration in the feedstock than used in conventional mass processes. This adaptation we have found enables the amount of maltose in the feedstock to react more completely and quickly in terms liquid hourly space velocity without generation of significant amounts of sorbitol or dextrose side products. As the sugar concentration of feedstock complements with the rate of reaction, the conversion of maltose to maltitol can be performed more thoroughly, while minimizing the amount of maltitol that is further degraded to glucose and sorbitol by further hydrogenation in the reactor.

Generation of side products increases with reaction time and also decreases the overall yield and purity of the desire product. In view of this problem associated with conventional batch reactor processes for making maltitol, a need exists for a better, more efficient, and economical way to achieve higher yields and less side products.

As often occurs with batch reactor systems, a certain amount of maltose feedstock can remain unreacted in the reactor. This situation may result from either the design or temperature process of a batch reactor, which can leave certain regions in the reactor isolated reactively. To overcome this problem, a conventional industrial response has been to increase the dwelling time of the batch so as to consume as much as possible of the feedstock in the reactor. A prolonged dwell time, however, often leads to side reactions that generate byproducts. The side reactions further breakdowns maltitol that has already formed into its constituents—glucose and sortitol—such as illustrated in Scheme B.

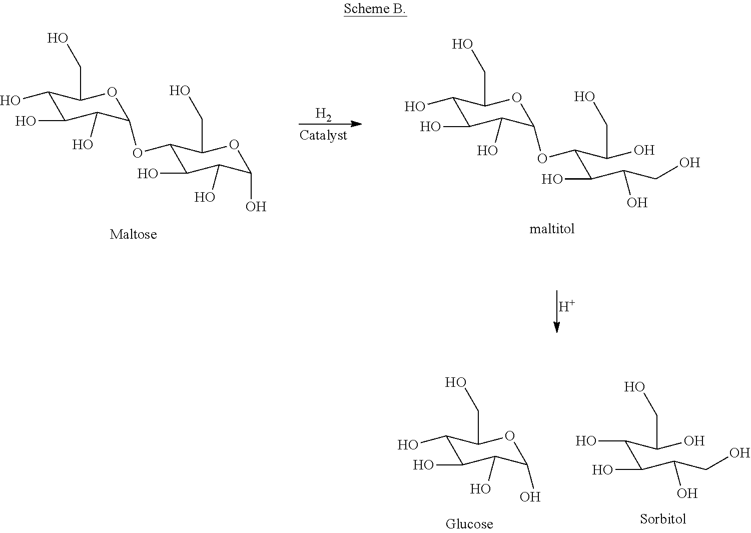

Another advantage of the present process is that it can reduce or eliminate the production of gluconic acid when converting aldehyde groups of the sugar molecules. Less acid in the solution can help prevent the leaching of catalysts, which can prolong the useful life of the catalysts and hydrogenation operations, and save costs for downstream processing operations such as waste disposal and product purification to remove catalyst metals that have dissolved. An associated benefit can include better quality maltitol product. The final product can contain an amount of leached catalyst metal that is at least 55% less relative to an amount of catalyst metal leached from a catalyst in a batch reactor process. In some embodiments, the amount of leached catalyst metal is less than 60%-85% (e.g., 65%, 67%, 70%, 72%, 75%, 78%, 80%, 83% or 84%) of an amount of catalysts that one may find leached from a catalyst in a conventional batch reactor process. The final product contains no more than about 3 ppm of a catalyst metal, typically ≤2.5 ppm, and desirably ≤2.0 ppm (e.g., 0.2 ppm, 0.5 ppm, ≤1.0 ppm, ≤1.5 ppm).

An insight to understanding the present process involves adjustments to space velocity, which can be expressed mathematically as SV≡υ0/V. In this expression, υ0 represents the volumetric flow rate of the reactants entering the reactor and V represents the volume of the reactor or catalyst bed. Space velocity values can be viewed as how many reactor volumes of feed can be treated in a unit time. As a method for relating the reactant liquid flow rate to the reactor volume at a standard temperature (given the mostly liquid nature of the feedstock in the present hydrogenation reaction) liquid hourly space velocity (LHSV) of the reactor system is used to indicate the number of reactor changes the system is undergoing in one hour. An operator can tune the volumetric flow rate of the reactants to optimize the hydrogenation reaction so as to generate the most efficient conversion of maltose to maltitol without overheating or over-reacting the maltose by letting it dwell too long in the reactor.

As the reactions in Example 1, below, show, a slow flow rate with longer reaction time helps maltose hydrogenation, but longer reaction time also makes more sorbitol. Hence, an advantage of the process is an ability to make a fine balance of these two factors—reaction time and flow rate.

According to the present process, one should maintain a LHSV of ≤3.0 per hour. In certain embodiments, for example, the LHSV is typically in a range from about 0.1 or 0.2 per hour to about 2.5 or 2.8 per hour (e.g., 0.4/h, 0.7/h, 2.3/h, or 2.7/h). In certain favored examples the reactant medium is processed through the reactor at a LHSV in a range from about 0.5 per hour to about 2.0 per hour (e.g., 0.6/h, 0.8/h, 1.0/hr, 1.2/h, 1.5/h. 1.7/h or 1.8/h).

In contrast to conventional hydrogenation processes that use a maltose feedstock having a high-concentration of more than 40 wt. % or 1.2 mol/L for making maltitol, the present process employs a lower maltose concentration of ≤30 wt. %. Typically, the maltose concentration is in a range from about 5 wt. % or 7 wt. % to about 26 wt. % or 28 wt. %, or any value or combination of values therein between. In particular embodiments, for instance, the maltose concentration of the feedstock can be about: 8 wt. %, 10 wt. %, 12 wt. % 15 wt. %, 17 wt. %, 18 wt. %, 20 wt. %, 23 wt. %, 25 wt. %, or 27 wt. %.

According to certain embodiments of the present process, the concentration of hydrogen and maltose substrate should be in a molar ratio of at least 20:1; typically, the molar ratio can be in a range from about 25:1 or 27:1 to about 55:1 or 60:1, or any value or combination of values therein between. For example, in certain embodiments, the molar ratio can be: ≥30:1, ≥32:1, ≥35:1, ≥40:1, ≥45:1, ≥48:1, or ≥50:1.

As the data in Example 2, below, demonstrates, maintaining higher pressures during the reaction are preferred to enable quick reaction of the sugar feed without degradation of either the product or catalyst. Reactions performed at lower pressure tend not to convert the sugars as efficiently nor yield products that are as clean as at higher pressures. Additionally, one also notices some degradation of and contamination from the catalyst at lower pressures.

During operations, the reaction pressure should be maintained relatively high so as to ensure sufficient concentration of hydrogen is soluble in an aqueous solvent matrix. The reaction pressure should be at least 1400 psi (˜10342.136 kPa). Typically, the reaction pressure is in a range from about 1600 psi (˜11031.612 kPa) or 1700 psi (˜11721.087 kPa) to about 2600 psi (˜17926.369 kPa) or 2700 psi (18615.845 kPa), or any value or combination of values therein between. For example, in certain embodiments, the pressure can be: 1800 psi (˜12410.563 kPa), 1900 psi (˜13100.039 kPa), 1950 psi (˜13444.777 kPa), 2000 psi (˜13789.515 kPa), 2100 psi (˜14478.990 kPa), 2150 psi (˜14823.728 kPa), 2200 psi (˜15168.466 kPa), 2270 psi (˜15651.099 kPa), 2300 psi (˜15857.942 kPa), 2350 psi (˜16202.680 kPa), 2400 psi (˜16547.417 kPa), 2450 psi (˜16892.155 kPa), 2500 psi (˜17236.893 kPa) or 2550 psi (˜17581.631 kPa).

According to certain embodiments, one can employ a fixed-bed reactor, which one can use a hydrogenation catalyst such as: Raney nickel, Raney copper, Raney copper on carbon, nickel on carbon, copper on carbon, and precious metals, including ruthenium, paladium, and platinum. As a particular embodiment, it is believed that a continuous process on nickel catalyst to make maltitol would be desirable.

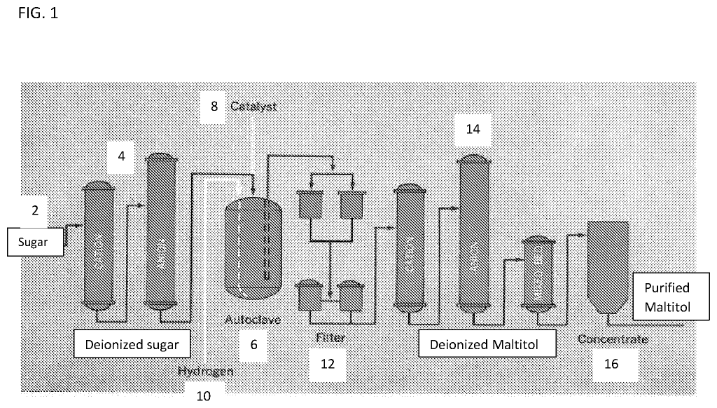

FIG. 1 depicts a schematic of a conventional batch reactor system. First, sugar is treated with cation 2 and anion 4 removal to deionize it. Second, the deionized sugar (maltose) is reacted in an autoclave or batch reactor 6 in the presence of a catalyst 8 with hydrogen 10. The resulting maltitol product mixture is then filtered 12, and the maltitol is deionized 14 and purified 16.

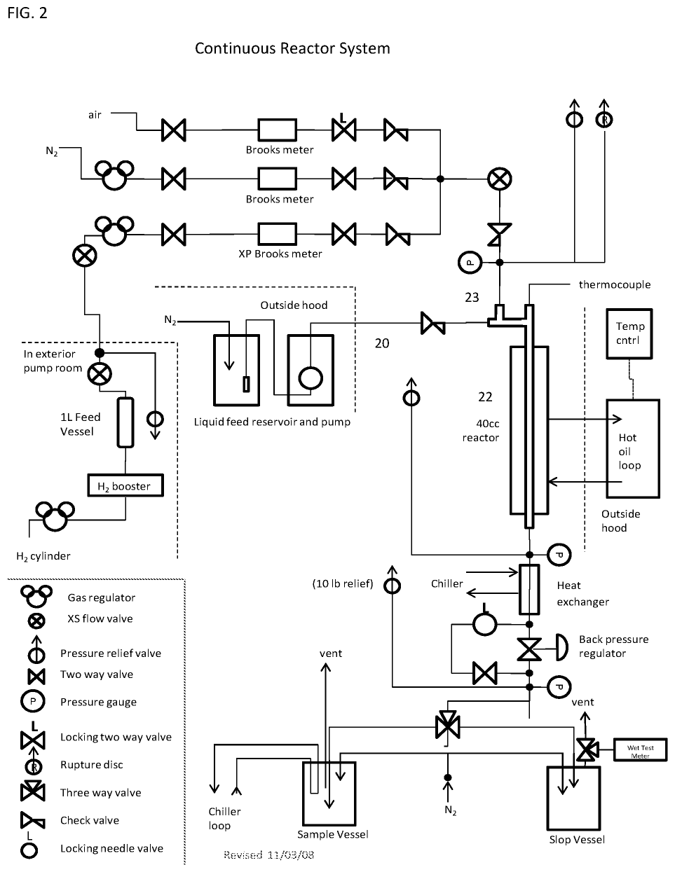

FIG. 2 depicts a continuous process reactor system according to an embodiment of the present invention. In general, a feedstock of sugar 20 is introduced continuously into a reactor 22. The concentration, rate of introduction, and dwelling time of the sugar feedstock is controlled to maintain optimal LHSV in the reactor so as to not over react the sugar with hydrogen. An advantageous feature is that a continuous reactor system affords better downstream separation without need for filtration.

### SECTION II—EXAMPLES

### Example 1

Raney Nickel from W.R. Grace was loaded into a 30 cubic centimeter fixed bed reactor, and hydrogen was thereafter supplied to the reactor at 1800 pounds per square inch, gauge, at a rate of 0.4 liters per minute, together with a liquid feed 30% maltose. The reactor temperature was at 140° C., and the liquid hourly space velocity was from 0.5-1 per hour.

TABLE 1
Maltose hydrogenation product concentrations
from 30 cc flow reaction; conditions below

| Sample Ex. | LHSV | maltitol | sorbitol | maltose | dextrose |
|---|---|---|---|---|---|
| 1 | 1 | 25.27 | 0.44 | 0.08 | 0.07 |
| 2 | 0.7 | 25.88 | 0.45 | 0.06 | 0.06 |
| 3 | 0.5 | 28.53 | 0.93 | nd | nd |
| 4 | 0.7 | 25.86 | 0.63 | 0.03 | 0.09 |

Units: %, Condition: 140° C., 1800 psi, H₂ flow rate: 0.4 L/min, 30% dry solids (DS), catalyst size 8 × 12 mesh (19538-62) with 1 ppm or less of Ni in a final product mixture.

### Example 2

Raney Nickel from W.R. Grace was loaded into a 30 cubic centimeter fixed bed reactor, and hydrogen was thereafter supplied to the reactor. The reaction was run respectively at 2000, 1100, and 100 pounds per square inch, gauge, at a rate of 0.8 liters per minute, together with a liquid feed 30% maltose. The reactor temperature was at 140 degrees Celsius, and the liquid hourly space velocity was from 0.5-1 hr⁻¹. As data summarized in Table 2 shows, one achieve a more efficient conversion of maltose to maltitol with minimal production of dextrose or sorbitol. The relatively faster and more complete reaction caused less leaching of the catalyst because of the generation of primarily gluconic acid. Hence, reactions at greater pressures help prevent nickel leaching. Conversely reactions at lower pressures also result in greater amounts of unreacted residual maltose.

TABLE 2
Maltose hydrogenation product concentrations from 30 cc flow reaction; conditions
below

| | | maltose | dextrose | maltitol | fructose | sorbitol | Ni (ppm) |
|---|---|---|---|---|---|---|---|
| Sample Ex. | pressure | g/kg | g/kg | g/kg | g/kg | g/kg | ppm |
| maltose feed | | 199.73 | 1.86 | 0.00 | 0.00 | 0.00 | 0 |
| 1 | 2000 psi | 0.39 | 2.07 | 142.54 | 0.43 | 4.42 | 0 |
| Comparative 1 | 1100 psi | 0.35 | 2.99 | 162.93 | 0.25 | 4.60 | 4.1 |
| Comparative 2 | 100 psi | 65.9 | 9.8 | 111.86 | 1.16 | 11.4 | 215 |

Units: %, Condition: 137° C., H₂ flow rate: 0.4 L/min, 20% dry solid (DS), catalyst size 6 × 8 mesh.

### Example 3

Into a 1 L Autoclave Engineers reactor were loaded 700 g of 30% maltose solution and 7 g of Raney nickel from W.R. Grace (19538-62). The reactor was purged 3 times with H₂, then heated up to 150° C., with 1400 psi of hydrogen. After 1 h and 4 h, reactor samples were pulled from the reactor to monitor reaction progress Smaller particle size of the catalyst material helps promote better maltose hydrogenation.

TABLE 3
Maltose hydrogenation products from a
1 L batch reaction; conditions below.

| | | maltose | maltitol | sorbitol |
|---|---|---|---|---|
| Sample Ex. | time | % | % | % |
| Feed | | 28.90 | 0.00 | 0.01 |
| 1 | 1 h | 18.50 | 10.00 | 0.34 |
| 2 | 4 h | 5.62 | 17.80 | 1.78 |

Unit: %, Condition: 150° C., 1400 psi, catalyst size 8 × 12 mesh (19538-62).

### Example 4

Into a 1 L Autoclave Engineers reactor were loaded 700 g of 30% maltose solution and 7 g of Raney nickel from W.R. Grace (19538-46). The reactor was purged 3 times with H₂, then heated up to 150° C., with 1400 psi of hydrogen. After 1 h and 4 h, reactor samples were pulled from reactor to monitor reaction progress. It is found that larger particle size of the catalyst material using the same amount of catalyst limits hydrogenation rate.

TABLE 4
Maltose hydrogenation products from a
1 L batch reaction; conditions below.

| | | maltose | maltitol | sorbitol |
|---|---|---|---|---|
| Sample Ex. | time | % | % | % |
| Feed | | 28.90 | 0.00 | 0.01 |
| 1 | 1 h | 18.11 | 10.64 | 0.40 |
| 2 | 4 h | 7.77 | 18.30 | 3.10 |

Units: % Conditions: 150° C., 1400 psi, catalyst size 3 × 6 mesh (19538-46).

### Example 5

Into a 1 L Autoclave Engineers reactor were loaded 700 g of 30% maltose solution and 7 g of Raney nickel from W.R. Grace (19538-62). The reactor was purged 3 times with H₂, then heated up to 140° C., with 1800 psi of hydrogen. After 1 h and 4 h, reactor samples were pulled from reactor to monitor reaction progress. The examples demonstrate that reactions performed at higher pressures help with more thorough hydrogenation while using the same amount of catalyst.

TABLE 5
Maltose hydrogenation products from a
1 L batch reaction; conditions below.

| | | maltose | maltitol | sorbitol |
|---|---|---|---|---|
| Sample Ex. | time | % | % | % |
| Feed | | 28.90 | 0.00 | 0.01 |
| 1 | 1 h | 8.27 | 18.40 | 0.65 |
| 2 | 4 h | 0.67 | 23.74 | 3.77 |

Units: % Conditions: 140° C., 1800 psi, catalyst size 3 × 6 mesh (19538-62).

Although the present invention has been described generally and by way of examples, it is understood by those persons skilled in the art that the invention is not necessarily limited to the embodiments specifically disclosed, and that modifications and variations can be made without departing from the spirit and scope of the invention. Thus, unless changes otherwise depart from the scope of the invention as defined by the following claims, they should be construed as included herein.

We claim:

1. A process for preparing maltitol comprising: reacting a medium containing maltose at a concentration of ≤30% with hydrogen in a continuous manner using a fixed-bed reactor at a reaction temperature and pressure sufficient to produce a final product containing a yield of maltitol of at least 90 mol. %, at a conversion rate in which no more than about 2 mol. % of said maltose concentration remains, and a sorbitol concentration of less than 1.0 mol. %.

2. The process according to claim 1, wherein a concentration of said hydrogen to maltose substrate is in a molar ratio of ≥20:1.

3. The process according to claim 2, wherein said molar ratio is ≥40:1.

4. The process according to claim 1, wherein said reaction temperature is in a range from about 100° C. to about 180° C.

5. The process according to claim 4, wherein said reaction temperature is in a range from about 110° C. to about 170° C.

6. The process according to claim 1, wherein said pressure is in a range from about 1400 psi to about 2700 psi.

7. The process according to claim 4, wherein said pressure is in a range from about 1800 psi to about 2500 psi.

8. The process according to claim 1, wherein said medium is processed in said reactor at a liquid hourly space velocity (LHSV) of ≤3.0 per hour.

9. The process according to claim 6, wherein said medium is processed through said reactor at a LHSV in a range from about 0.2 to 2.8 per hour.

10. The process according to claim 1, wherein a dextrose concentration is controlled to be less than 0.20 mol. % in said final product.

11. The process according to claim 1, wherein said sorbitol concentration is less than 0.8 mol %.

12. The process according to claim 1, wherein said fixed-bed reactor has a catalyst selected from the group consisting of: Raney nickel, Raney copper, Raney copper on carbon, nickel on carbon, copper on carbon, and precious metals, including ruthenium, paladium, and platinum.

13. The process according to claim 1, wherein said final product contains an amount of leached catalyst that is at least 55% less relative to an amount leached from a catalyst in a batch reactor process.

14. The process according to claim 1, wherein said final product contains no more than about 3 ppm of a catalyst.

15. A process for making maltitol comprising: performing hydrogenation on a medium containing a concentration of maltose in a continuous manner using a fixed-bed reactor; controlling said hydrogenation to generate a sorbitol concentration of less than 0.85 mol. % in a final product and consuming at least 98% of said maltose within said reactor at a liquid hourly space velocity (LHSV) in a range from about 0.2 to 2.5 per hour.

16. The process according to claim 14, wherein said sorbitol concentration is maintained at less than 0.3 mol. %.

17. The process according to claim 1, wherein a dextrose concentration is controlled to be less than 0.3 mol. % in said final product.
